(19) United States
(12) Patent Application Publication
(10) Pub. No.: US 2019/0033294 A1
(43) Pub. Date: Jan. 31, 2019

(54) METHODS OF SCREENING DRUGS FOR CANCER TREATMENT USING CELLS GROWN ON A FIBER-INSPIRED SMART SCAFFOLD

(71) Applicants: UNIVERSITY OF SOUTH FLORIDA, Tampa, FL (US); TRANSGENEX NANOBIOTECH, INC., Lutz, FL (US)

(72) Inventors: Subhra MOHAPATRA, Lutz, FL (US); Shyam S. MOHAPATRA, Lutz, FL (US); Rajesh R. NAIR, Tampa, FL (US)

(21) Appl. No.: 16/075,739

(22) PCT Filed: Feb. 17, 2017
(86) PCT No.: PCT/US2017/018506

§ 371 Date: Aug. 6, 2018

Related U.S. Application Data
(60) Provisional application No. 62/296,847, filed on Feb. 18, 2016.
(60) Provisional application No. 62/297,710, filed on Feb. 19, 2016.

(51) Int. Cl. G01N 33/50

(52) U.S. Cl. CPC G01N 33/5011; G01N 2500/10; G01N 33/5082; G01N 33/502

(57) ABSTRACT

Described are methods of screening drugs for cancer treatment using a fiber-inspired smart scaffold cell culture system. The system recapitulates the actual in vivo tumor microenvironment, thereby ensuring efficacy in clinical trials by identifying drugs that will be effective in treating cancer. The drugs identified by the system may then be used to treat cancers, including breast cancer and colorectal adenocarcinoma. In addition, this screening system provides a platform for methods relating to the personalized treatment of cancer.

### CROSS-REFERENCE TO RELATED APPLICATIONS

This application claims priority to U.S. Provisional Application No. 62/296,847 filed on Feb. 18, 2016, and U.S. Provisional Application No. 62/297,710 filed on Feb. 19, 2016, which are incorporated fully herein by reference.

### TECHNICAL FIELD

The present disclosure relates to methods of screening for drugs for the treatment of cancer. Specifically, the invention relates to methods of using tumoroids grown on a nanofiber scaffold platform for screening potential anticancer drugs for treatment of cancer. The present disclosure also relates to screening for drugs for the treatment of breast cancer and colorectal adenocarcinoma.

### BACKGROUND

Cancer consistently ranks as one of the most common causes of death worldwide, and in the United States, is the second most common cause of death, accounting for nearly 1 of every 4 deaths. Cancer arises from a single cell that has transformed from a normal cell into a tumor cell. Such a transformation is often a multistage process, progressing from a pre-cancerous lesion to malignant tumors. Multiple factors contribute this progression, including aging, genetic contributions, and exposure to external agents such as physical carcinogens (e.g., ultraviolet and ionizing radiation), chemical carcinogens (e.g., asbestos, components of tobacco smoke, etc.), and biological carcinogens (e.g., certain viruses, bacteria, and parasites). Prevention, diagnosis and treatment of cancer may take many different forms. Treatment may include chemotherapy, radiation therapy, and surgical removal of a tumor or cancerous tissue. Despite the availability of numerous prevention and treatment methods, such methods often meet with limited success in effectively preventing and/or treating the cancer at hand due to the inherent heterogeneity and propensity for development of drug resistance. Accordingly, a need exists for the identification of compositions and methods for the prevention and/or treatment of cancer to facilitate clinical management and prevention against progression of disease.

### SUMMARY

In one aspect, disclosed is a method of screening drugs for cancer treatment, the method comprising: a) growing target cancer cells on a three-dimensional scaffold of fibers, wherein said fibers are formed from a mixture comprising a ratio polyethylene glycol-polylactic acid block copolymer (PEG-PLA) and a poly(lactic-co-glycolic acid) (PLGA); b) contacting at least one candidate cancer drug to the cells; and c) measuring IC₅₀ values of the at least one cancer drug.

### BRIEF DESCRIPTION OF THE DRAWINGS

FIG. 1A-C shows the characterization of the scaffolds. (A) FTIR of mPEG and mPEG-PLA. (B) 1HNMR of mPEG-PLA. (C) SEM of PLGA and 3P scaffolds.

FIG. 2A-D shows stem cell characterization of cells growing in monolayer versus tumoroids. (A) MCF-7 cells were plated on the FiSS platform (Scaffold) for 6 days and the resulting first-generation tumoroids were visualized using Nuc-blue. (B) Single cell suspension of the MCF-7 tumoroids were stained with APC-Cy7 conjugated CD44 antibody and APC conjugated CD24 antibody and the CD44^{high} CD24^{low} cells were detected using flow cytometry and analyzed using FlowJo software. (C-D) MCF-7 and MCF-7/Dox cells were grown as a monolayer in cell culture dish or as a tumoroid on the scaffold. After 6 days in culture, the monolayer and the tumoroids were harvested and processed for RNA and protein extraction. The extracted RNA was subjected to qRT-PCR and the extracted protein was subjected to western blotting. HPRT transcript was used for normalization of qRT-PCR data and HPRT protein was used as loading control for the western blot. Data are represented as Mean±SEM of three independent experiments.

FIG. 3A-B shows that tumoroids show increased drug resistance compared to cells grown in monolayer. (A) MCF-7 and MCF-7/dox cells were cultured on the scaffold for 6 days and then visualized using Nuc-blue. (B) MCF-7 and MCF-7/dox cells were cultured on or on scaffold for 6 days and then cells were treated with increasing concentration of Doxorubicin. Forty-eight hrs post-treatment, cell viability was analyzed using presto-blue as per manufacturer's instructions. The % cell death was analyzed and the IC₅₀ calculated using Graph Pad Prism. Data are represented as Mean±SEM of three independent experiments performed in triplicates (*p<0.05).

FIG. 4A-B shows cobalt chloride infused scaffold induces hypoxia in tumoroids. (A) MCF-7 and MCF-7/dox cells were cultured on the scaffold containing 50 μM COCl2 for 6 days and then visualized using Nuc-blue. Images were captured using a fluorescence microscope and the diameter of the tumoroids were analyzed using ImageJ. (B) Six day old tumoroids grown on cobalt chloride-infused scaffold were stained with hypoxic dye as per the manufacturers. Data are represented as Mean±SEM of three independent experiments performed in triplicates (*p<0.05).

FIG. 5 shows lactate concentration measured from the deproteinized media using an L-Lactate Assay Kit. Lactate concentration was measured in both monolayer and scaffold culture condition in both MCF7-WT and MCF7-DOX. Concentration was expressed as nmole/mg protein.

FIG. 6A-B shows high-through put screening of breast cancer cells in monolayer. (A) MCF-7 and (B) MCF-7/dox cells were cultured on monolayer and then treated with increasing concentration of compounds from NCI Approved Oncology Drugs Set VII. Forty-eight hrs post-treatment, cell viability was analyzed using presto-blue as per manufacturer's instructions. The % cell death was analyzed and the IC₅₀ calculated using Graph Pad Prism. Data are represented as Mean of experiments performed in triplicates.

FIG. 7 shows that Actinomycin D is effective in inducing cell death in tumoroids. MCF-7 and MCF-7/dox cells were cultured on monolayer or on scaffold in normoxia or hypoxia for 6 days. At the end of culture period, the monolayer cells and tumoroids were treated with increasing concentration of Actinomycin D. Forty-eight hrs post-treatment, cell viability was analyzed using presto-blue as per manufacturer's instructions. The % cell death was analyzed and the IC₅₀ calculated using Graph Pad Prism. Data are represented as Mean±SEM of three independent experiments performed in triplicates (*p<0.05).

FIG. 8A-D shows that Actinomycin D treatment reduced cell viability and the CD44+/24− sub-population in MCF-7 by downregulating Sox2. MCF-7 and MCF-7/dox were allowed to form tumoroids on scaffold for 6 days following which they were exposed to Actinomycin D for 24 hrs. At the end of 24 hrs the tumoroids were dissociated and processed for either western blotting (A) or real-time RT-PCR (B). Sox2 protein and transcript levels were analyzed and normalized to HPRT (C) Second generation MCF-7 tumoroids were cultured in a 96 well plate for 6 days. Tumoroids were treated with increasing concentrations of Actinomycin D on day 4 in groups of 4 wells. After a 48 hr treatment, cell viability was assayed using Cell Titer Glo(Promega) on day 6. Viability was calculated as a percent of control untreated wells and graphed using Graph Pad Prism software. (D) Tumoroids were treated with Actinomycin D for 48 hr. Following treatment tumoroids were dissociated and cells were co-stained using FITC conjugated CD44 antibody and Alexa Fluor-647 conjugated CD24 antibody. CD44^{high}CD24^{low} stem-like populations in MCF7 tumoroids were assessed by flow cytometer. Data was collected using a BD FACS Canto 2 flow cytometer.

FIG. 9A-B shows a series of images depicting that breast cancer cells, irrespective of its drug sensitivity, grow to form resistant tumoroids on the scaffold.

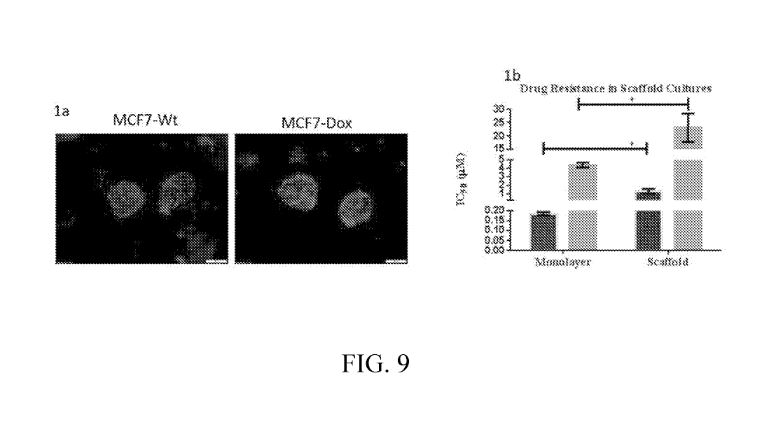

FIG. 10A-D shows a series of images depicting breast cancer tumoroids, irrespective of its drug sensitivity, show induction in transcription factors and cell surface receptors involved in maintenance of sternness.

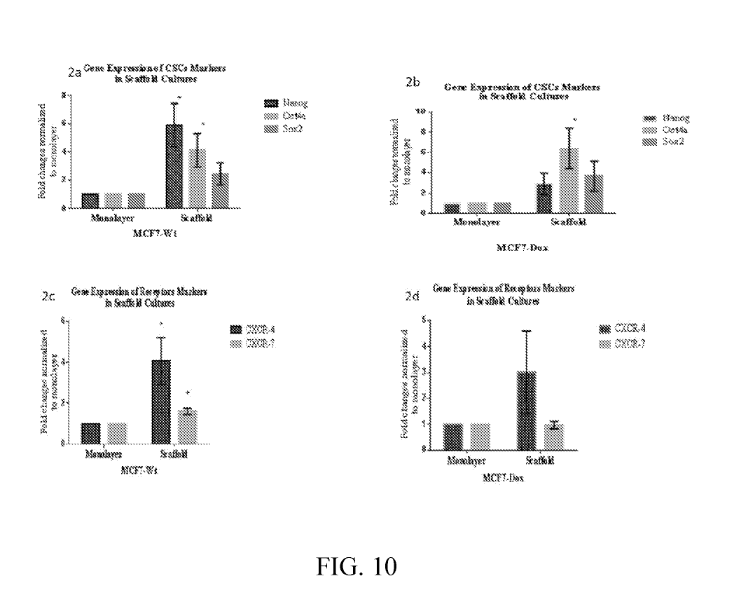

FIG. 11 shows a table depicting a comparison of the IC₅₀ values in Breast cancer grown as a monolayer or tumoroids.

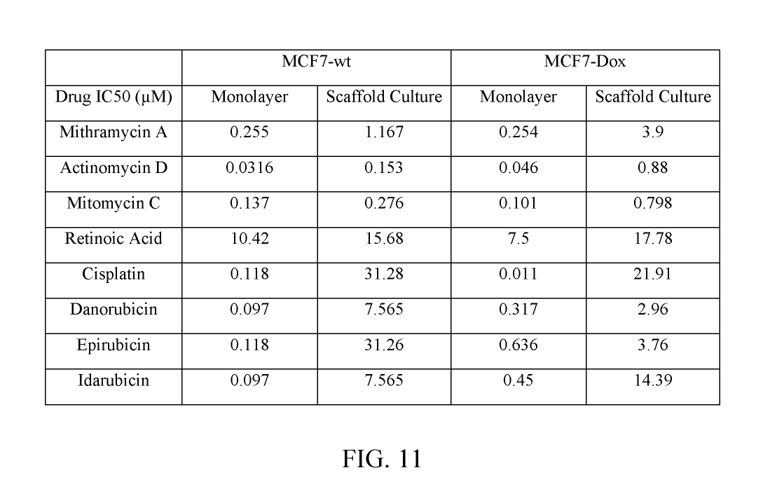

FIG. 12 shows is a table depicting screening of clinically approved drugs in a panel of breast cancer cells (Values are calculated IC₅₀).

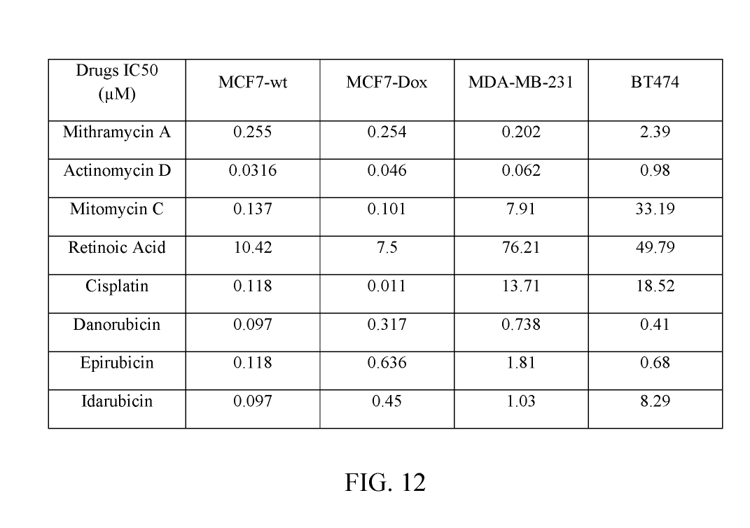

FIG. 13A-B shows a series of images depicting HT-29 cells cultured on the 3D fibrous scaffold for 7 days were stained with DAPI and imaged using the fluorescence microscope. (A) Magnification: 4× (B) Magnification: IOX.

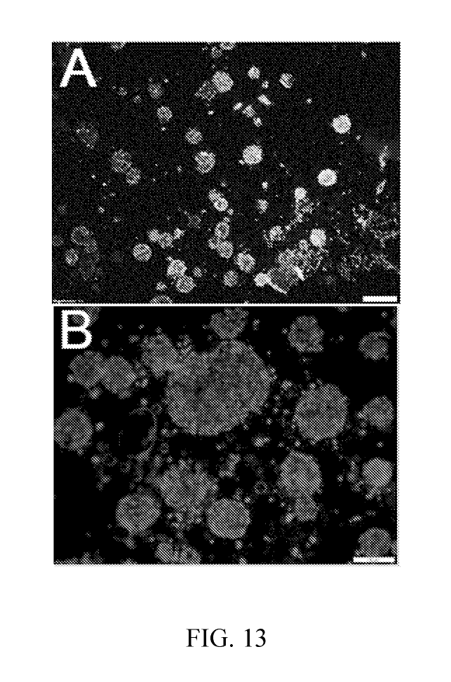

FIG. 14A shows a series of images depicting a representative graph of one trial of monolayer culture and treatment. HT-29 cells were cultured in 2D monolayer for 24 hours. After 24 hours, the nine FDA-approved anticancer drugs were individually added to the cells at concentrations of 0.1, 1, 10, and 20 μM, and the cells were incubated for 72 hours, at which point viability of the cells was quantitated using Presto Blue assay (Life Technologies) according to the manufacturer's protocol. Raw fluorescence values of the treated groups were normalized to the average of the control and graphed in GraphPad Prism.

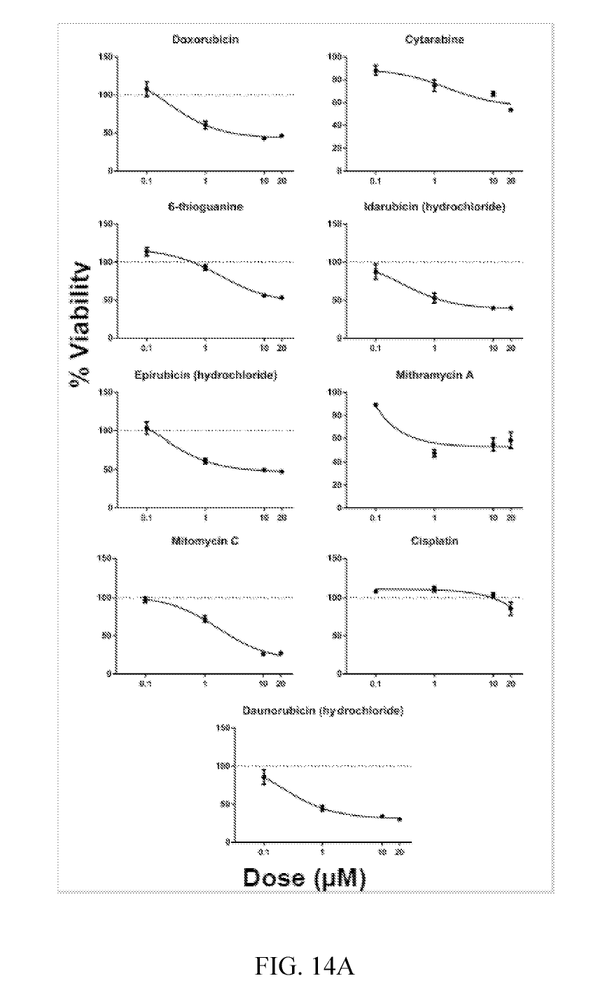

FIG. 14B shows a series of images depicting a representative graph of one trial of culture and treatment on the fibrous scaffold HT-29 cells were cultured in the 3D fibrous scaffold from day 1 to day 7, at which point the nine FDA-approved anticancer drugs were individually added to the cells at concentrations of 0.1, 1, 10, and 20 μM. The cells were incubated for an additional 72 hours, at which point viability of the cells was quantitated using Celltiter-Glo assay (Promega) according to the manufacturer's protocol. Raw luminescence values of the treated groups were normalized to the average of the control and graphed in GraphPad Prism.

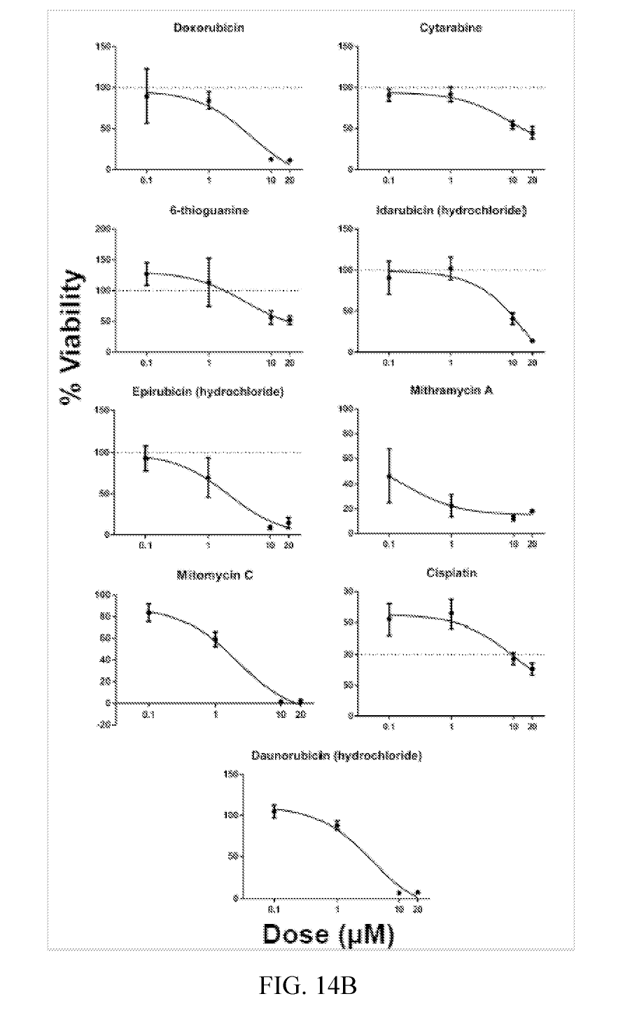

FIG. 15 shows an image depicting IC₅₀ values were calculated using GraphPad Prism for each of the nine anticancer drugs used to inhibit cell viability of HT-29 cells grown on monolayer and fibrous scaffold culture. IC₅₀ values from three trials were averaged for each culture method of the nine drugs and plotted as mean±standard deviation.

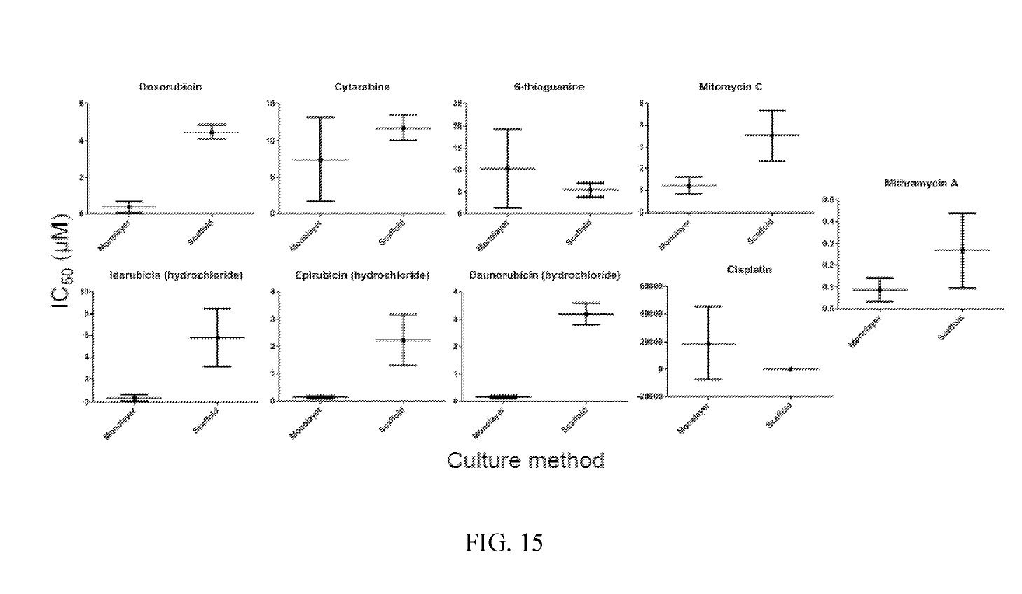

FIG. 16A-B shows a series of images depicting (A) A representative graph of one trial of monolayer culture and treatment. HT-29 cells were cultured in 2D monolayer for 24 hours. After 24 hours, the nine FDA-approved anticancer drugs were individually added to the cells at concentrations of 0.1, 1, 10, and 20 μM, and the cells were incubated for 72 hours, at which point viability of the cells was quantitated using Presto Blue assay (Life Technologies) according to the manufacturer's protocol. Raw fluorescence values of the treated groups were normalized to the average of the untreated controls and graphed in GraphPad Prism. (B) A representative graph of one trial of culture and treatment on the fibrous scaffold. HT-29 cells were cultured in the 3D fibrous scaffold from day 1 to day 7, at which point the nine FDA-approved anticancer drugs were individually added to the cells at concentrations of 0.1, 1, 10, and 20 μM. The cells were incubated for an additional 72 hours, at which point viability of the cells was quantitated using Celltiter-Glo assay (Promega) according to the manufacturer's protocol. Raw luminescence values of the treated groups were normalized to the average of the untreated controls and graphed in GraphPad Prism.

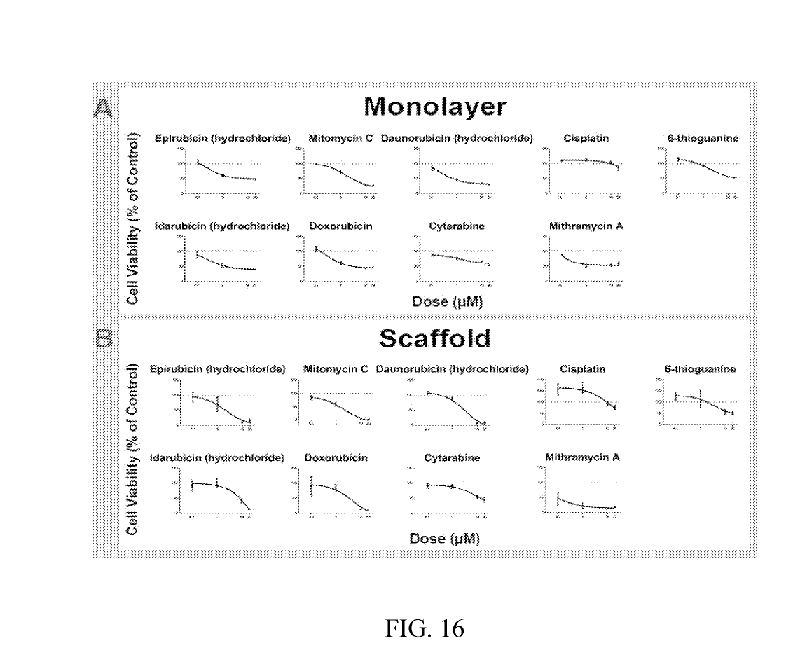

FIG. 17 shows an image depicting IC₅₀ values were calculated using GraphPad Prism for each of the nine anticancer drugs used to inhibit cell viability of HT-29 cells grown on monolayer and on fibrous scaffold culture. IC₅₀ values from three trials were averaged for each culture method of the nine drugs and plotted as mean±standard error of the mean.

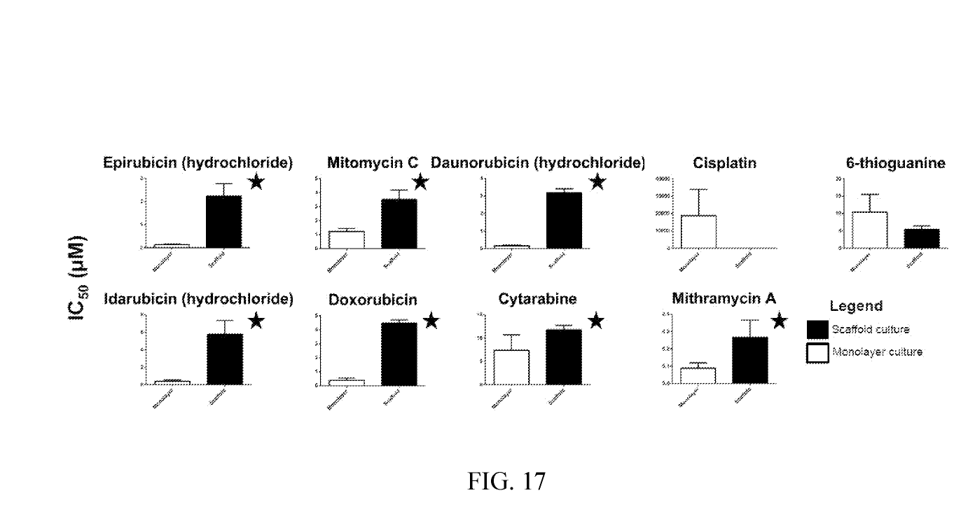

### DETAILED DESCRIPTION

Disclosed herein are methods for screening drugs for the treatment of cancer. The disclosed methods use a cell culture system that mimics in vivo tumors. This system may be used to screen for anti-cancer drugs. Thereby ensuring efficacy in clinical trials. In addition, such systems provide a methods relating to the personalized treatment of cancer. There is high rate of attrition in drugs used in the clinic for treating cancer. One of the main culprits for this attrition is the use of screening platforms that do not mimic the actual in vivo tumor microenvironment. For example, there are in vivo factors that are not accounted for in traditional in vitro methods (e.g., oxygen levels, glucose levels, and pH levels). A fiber-inspired smart scaffold (FiSS) 3D cell culture system is described herein. This FiSS culture system allows for the growth of cancer cell lines, tumor biopsies, and co-cultured cells using standard cell culture techniques, and cell culture wares, for example. The cells grow as three-dimensional tumoroids that mimic the growth of in vivo tumors. For example, (i) the tumoroids may be more resistant to cell death than cells grown on monolayer; (ii) the tumoroids may express markers of epithelial mesenchymal transition and; (iii) the tumoroids may maintain cancer stem cell populations. In addition, the FiSS 3D culture system is conducive for use with high throughput screening assay platforms for the detection of drugs, which will lead to successfully identifying drugs that will have a higher success rate for the treatment for cancer.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . N, or combinations thereof” are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, the terms “cancer,” “cancer cells,” “neoplastic cells,” “neoplasia,” “tumor,” and “tumor cells” (used interchangeably) refer to cells which exhibit relatively autonomous growth so that they exhibit an aberrant growth phenotype characterized by a significant loss of control of cell proliferation (i.e., de-regulated cell division). These cells can be malignant or benign.

The terms “cell,” “cell line,” and “cell culture” include progeny. It is also understood that all progeny may not be precisely identical in DNA content due to deliberate or inadvertent mutations. Variant progeny that have the same function or biological property, as screened for in the originally transformed cell, are included.

An “effective amount” is an amount sufficient to effect beneficial or desired results. An effective amount can be administered in one or more administrations, applications, or dosages.

As used herein, “scaffold” refers to a three-dimensional porous sold biomaterial that may: (1) promote cell-biomaterial interactions, cell adhesion, and extracellular matrix deposition; (2) may permit sufficient transport of gasses, nutrients, and/or regulatory factors to allow cell survival, proliferation, and/or differentiation; (3) may be biodegrade at a controllable rate that approximates the rate of tissue regeneration under culture conditions of interest; and/or (4) may provoke a minimal degree of inflammation or toxicity if introduced in vivo.

The terms “fiber-inspired smart scaffold” and “FiSS” and “fibrous scaffold” and “nano-fiber scaffold” as used herein, may be used interchangeably.

The term “PLGA” refers to poly(lactic-co-glycolic acid) that is synthesized by means of random ring-opening co-polymerization of two different monomers, the cyclic dimers (1,4-dioxane-2,5-diones) of glycolic acid and lactic acid. Depending on the ratio of lactide to glycolide used for the polymerization, different forms of PLGA can be obtained: these are usually identified in regard to the monomers' ratio used (e.g. PLGA 75:25 identifies a copolymer whose composition is 75% lactic acid and 25% glycolic acid).

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially.

“Subject” as used herein can mean a mammal that wants to or is in need of being treated for cancer. The mammal can be a human, chimpanzee, dog, cat, horse, cow, mouse, or rat.

The term “tumoroid” as used herein, refers to a micrometastatic compact aggregate of tumor cells. Tumoroids can respond to the same biochemical, nanotopographical, and mechanical cues that drive tumor progression in the extracellular matrix.

The term “IC₅₀” as used herein, is a measurement that may represent the halfway point in which a compound of interest may produces complete inhibition of a biological or biochemical function (e.g., metabolism). This information may be derived based on pharmacological data in reference to a dose-response curve. As the dosage of an inhibitory compound is increased, the biological function it affects may decrease, and the point at which the concentration of the inhibitor has suppressed 50% of the biological activity may be referred to as the IC₅₀. IC₅₀ may be used as a measurement of antagonist, or inhibitory drug potency, as well as a quantification of the toxicological effects of inhibitory compounds.

The terms “synergistic” and “synergism” as used herein, refers to drug combinations in which the drugs potentiate the effects of each other.

The term “parenterally,” as used herein, refers to modes of administration which include intravenous, intramuscular, intraperitoneal, intrasternal, subcutaneous and intraarticular injection and infusion.

The terms “chemotherapeutic agent” and “anti-cancer drug” and “drug for the treatment of cancer” and as used herein, may be used interchangeably.

The term “control”, as used herein, is an alternative subject or sample used in an experiment for comparison purpose and included to minimize or distinguish the effect of variables other than an independent variable.

The term “positive control” as used herein, refers to a “control” that is designed to produce the desired result, provided that all reagents are functioning properly and that the experiment is properly conducted.

The term “negative control” as used herein, refers to a “control” that is designed to produce no effect or result, provided that all reagents are functioning properly and that the experiment is properly conducted. Other terms that are interchangeable with “negative control” include “sham,” “placebo,” and “mock.”

The term “culturing” as used herein, refers to maintaining cells under conditions in which they can proliferate and avoid senescence as a group of cells. “Culturing” can also include conditions in which the cells also or alternatively differentiate.

The term “stem cell” as used herein, refers to any self-renewing totipotent, pluripotent cell or multipotent cell or progenitor cell or precursor cell that is capable of differentiating into multiple cell types.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. METHOD OF SCREENING

The present invention is directed to methods for screening pharmaceuticals for cancer treatment efficacy using FiSS. The cells grow as a three-dimensional tumoroid that mimics the growth of the in vivo tumors. For example, (i) the tumoroids are more resistant to cell death that cells grown on monolayer; (ii) the tumoroids show markers of epithelial mesenchymal transition and; (iii) the tumoroids show a healthy maintenance of cancer stem cell population. The system can be used to successfully identify drugs that will have a higher success rate of performing efficiently in the clinic for the treatment for cancer.

The present invention includes a method of screening drugs for cancer treatment, the method comprising of growing cells on a three-dimensional scaffold of randomly oriented fibers, wherein said fibers are formed from a mixture comprising a ratio polyethylene glycol-polylactic acid block copolymer (PEG-PLA) and a poly(lactic-co-glycolic acid) (PLGA); contacting at least one cancer drug to the cells; and measuring IC₅₀ values of the at least one cancer drug.

a. Fiber-Inspired Smart Scaffold (FiSS)

In the disclosed methods, the FiSS is seeded with cancer cells and tumoroids are allowed to form. The FiSS seeded with cancer cells is contacted with one or more drugs for a given period of time. Any number of cell characteristics may be measured before, during, or after contact with a drug. For example, cell number and/or cell morphology may be measured, counted, or analyzed. Dead and live cancer cells may then be quantitated and the efficacy of the drug for cancer treatment is determined. The FiSS system can be used in a 96 well format and may be compatible with one or more commercially available kits for cell death analysis in colorimetric, fluorometric, and luminescent read outs.

i. Composition of the Scaffold

The chemical structure of PEG is H—(O—CH₂—CH₂)n-OH. PEG is also known as polyethylene oxide (PEO) or polyoxyethylene (POE), depending on its molecular weight. PEG usually refers to oligomers and polymers with a molecular mass below 20,000 g/mol. PEGs are prepared by polymerization of ethylene oxide and are commercially available over a wide range of molecular weights from 300 g/mol to 10,000,000 g/mol. Different forms of PEG are also available, depending on the initiator used for the polymerization process the most commoninitiator is a monofunctional methyl ether PEG, or methoxypoly(ethylene glycol), abbreviated mPEG. Lower-molecular-weight PEGs are also available as purer oligomers, referred to as monodisperse, uniform, or discrete. In some embodiments, the PEG used to prepare the 3P and 3PC scaffolds described herein is a monomethoxy glycol (mPEG) having a molecular weight between approximately 0.5 and 20 kDa. Included herein are embodiments wherein the molecular weight of the PEG or mPEG is approximately 0.5, 1, 2, 3, 4, 5, 6, 7, 8, 9, 10, 11, 12, 13, 14, 15, 16, 17, 18, 19, or 20 kDa. In one embodiment, the molecular weight of PEG is approximately 2 kDa.

Polylactic acid or polylactide (PLA) ((C₃H₄O₂)n) is a thermoplastic aliphatic polyester derived from renewable resources, such as corn starch, tapioca roots, chips or starch, or sugarcane. Polymerization of a racemic mixture of L- and D-lactides usually leads to the synthesis of poly-DL-lactide (PDLLA), which is amorphous. Use of stereospecific catalysts can lead to heterotactic PLA which has been found to show crystallinity. The degree of crystallinity is largely controlled by the ratio of D to L enantiomers used, and to a lesser extent on the type of catalyst used. Due to the chiral nature of lactic acid, several distinct forms of polylactide exist: poly-L-lactide (PLLA) is the product resulting from polymerization of L,L-lactide (also known as L-lactide). PLLA has a crystallinity of around 37%, a glass transition temperature between 60-65° C., a melting temperature between 173-178° C. and a tensile modulus between 2.7-16 Gpa. Accordingly, the 3P and 3PC scaffolds provided herein can comprise a PLA composition having only D-enantiomers, only L-enantiomers or a mixture of D- and L-enantiomers. In some embodiments, the PLA composition used to prepare the 3P or 3PC composition contains a racemic mixture of D- and L-enantiomers. PLGA is synthesized by means of random ring-opening co-polymerization of two different monomers, the cyclic dimers (1,4-dioxane-2,5-diones) of glycolic acid and lactic acid.

Common catalysts used in the preparation of this polymer include tin(II) 2-ethylhexanoate, tin(II) alkoxides, or aluminum isopropoxide. During polymerization, successive monomeric units (of glycolic or lactic acid) are linked together in PLGA by ester linkages, thus yielding a linear, aliphatic polyester as a product. Depending on the ratio of lactide to glycolide used for the polymerization, different forms of PLGA can be obtained: these are usually identified in regard to the monomers' ratio used (e.g. PLGA 75:25 identifies a copolymer whose composition is 75% lactic acid and 25% glycolic acid). In one embodiment, the PLGA contains approximately 85% lactic acid and 15% glycolic acid. Also included herein are embodiments, where the lactic acid:glycolic ratio of PLGA is approximately 75:25, 80:20, 85:15, 90:10, and 95:5.

In some embodiments, the 3P scaffold is composed predominantly of poly(lactide-co-glycolide) (PLGA) random copolymer and a poly-lactide-poly(ethylene glycol) (PLA-PEG) block copolymer. In certain further embodiments, the 3P scaffold also comprises chitosan. The chitosan can be coated onto the 3P scaffold. Chitosan coated scaffolds are referred to herein 5 as 3PC scaffolds. The fiber polymer can be constructed by open ring polymerization of mPEG and PLA mixed with PLGA and electrospun. Both PLGA and PLA are used extensively in electrospinning for tissue engineering and drug delivery applications because they possess good mechanical properties, controlled degradability, and excellent biocompatibility (Zhou H, et al., Acta biomaterialia, 8:1999-2016 (2012), Xin X J, et al., Biomaterials, 28:316-325 (2007), and Kim K. et al., Biomaterials, 28:316-25 (2007)). Incorporation and controlled release of a hydrophilic antibiotic using poly(lactide-co-glycolide)-based electrospun nanofibrous scaffolds (Kim K. et al., Journal of Controlled Release, 98:47-56 (2004)). PEG is used to modify and enhance the hydrophilicity of the fibers; in addition it is nontoxic and non-immunogenic. PEG's protein-resistant properties arise from imparted nonionic charges, and a high excluded volume which facilitate steric repulsion thus minimizing the adsorption of proteins. Typical methods for spheroid formation employ similar non adherent surface modifications.

In some embodiments, the ratio of PEG-PLA to PLGA in each scaffold fiber is approximately 1:4. In other embodiments, the ratio of PEG-PLA to PLGA in each scaffold fiber is approximately 1:10. In still other embodiments, the ratio of PEG-PLA to PLGA in each scaffold fiber is approximately 1:2, 1:3, 1:4, 1:5, 1:6, 1:7, 1:8, 1:9, 1:10, or 1:20.

The PEG-PLA and PLGA can be formed into fibers via any method known to those of skill in the art. In some embodiments, solutions of PEG-PLA and PLGA are electrospun to form PEG-PLA-PLGA fibers. The scaffold fibers can be electrospun at any voltage, flow rate, and distance that provide for a fiber diameter between approximately 0.3 and 10 μm, or more preferably a fiber diameter between approximately 0.69 to 4.18 μm. In one embodiment, solutions of PEG-PLA and PLGA are electrospun at a positive voltage of 16 kV at a flow rate of 0.2 ml/hour and a distance of 13 cm using a high voltage power supply. The fibers may be collected onto an aluminum covered copper plate at a fixed distance of approximately 70 μm. The present invention further includes a 3P or 3PC scaffold prepared by collecting the electrospun fibers at a fixed distance between approximately 60 μm and 80 μm.

The resulting 3P or 3PC scaffold is a three-dimensional fibrous scaffold having pores. In some embodiments, the scaffold comprises pores having a diameter of less than approximately 20 μm. In other embodiments, the 3P or 3PC scaffold comprises pores having a diameter of less than approximately 15, 10 or 5 μm.

ii. Fabrication of the Scaffold

The scaffold can be fabricated by any suitable technique or method. Such techniques and methods include, without limitation, electrospinning, solvent casting/salt leaching, ice particle leaching, gas foaming/salt leaching, solvent evaporation, freeze drying, thermally induced phase separation, micromolding, photolithography, microfluidics, emulsification, decellularization processes, self-assemblies, microfiber wet spinning, melt-blown processing, sponge replication methods, simple calcium phosphate coating methods, inkjet printing, melt-based rapid prototyping processing or a combination thereof. One of skill in the art will appreciate that the technique(s) or method(s) used for scaffold fabrication will vary depending on, inter alia, the components present in the scaffold.

iii. Scaffold Materials

Scaffold materials can be synthetic, biologic, or combinations thereof. The scaffold materials can be degradable or non-degradable. The scaffold materials can be biocompatible. Synthetic scaffold materials can include, without limitation, PLA, PLG, PLGA, and PHA, PLLA, PGA, PCL, PDLLA, PEE based on PEO, and PBT.

b. Cells

Cells for use in the methods described herein may include, but are not limited to, cell lines and co-cultured cells. Cells for use in the methods described herein can include, but are not limited to, cancer cell lines, cells obtained from tumor biopsies, or co-cultured cells (e.g., growth of more than one distinct cell type in a single culture). The tumor biopsy may be from a subject prior to treatment for cancer or from a subject undergoing treatment for cancer. The biopsy may be from a subject not responding to cancer treatment. In some embodiments, the tumor biopsy is from a patient with breast cancer. In some embodiments, the tumor biopsy is from a patient with colorectal adenocarcinoma.

In some embodiments, the cells are mammalian. In some embodiments, the cells can be human, mouse, rat, monkey, dog. Examples of cells to be used in the methods disclosed herein can include, but are not limited to, MCF7 (ER+, Her2−), BT474 (ER+, Her2+), MDA MB-231 (ER−, PR− Her2−), 600MPE, AU565, BT-20, BT-474, BT-483, BT-549, EVSA-T, Hs578T, SkBr3, T-47D, Hs 190.T, Hs 344.T, Hs 350.T, Hs 841.T, Hs 849.T, Hs 851.T, Hs 861.T, MDA-MB-453, MDA-kb2, MB157, BT-20, Hs 578Bst, Hs 578T, Hs 579.Mg, ZR-75-1, ZR-75-30, T47D-KBluc, T-47D, MDA-MB-134-VI, MDA-MB-175-VII, BT-474, UACC-812, BT-483, BT-549, Hs 574.T, UACC-893, HCC38, HCC70, HCC202, HCC1008, HCC1143, HCC1187, HCC1395, HCC1419, HCC1500, HCC1599, HCC1937, HCC1954, HCC2218, UACC-2648, MCF 10A, MCF 10F, MCF 10-2A, UACC-3199, Hs 564(E).Mg, Hs309.T, UACC-3133, UAC-732, MDA-MB-157, HCC1569, SW527 [SW 527, SW-527], Hs 742.T, CMMT, NMuMG, 4T1, MM3MG, JC, MMT 060562, +/+MGT, MM2MT, RIIIMT, EMT6, CL-S1, CSMαβ6C [CSMab6C], CMH1α, CMαβ1h, C127I, MMSMTC, Rn2T, 13762 MAT B III, NMU, RBA, Hs 741.T, Rn1T, LA7, CF37.Mg, CF38.Mg, CF41.Mg, CF34.Mg, HT-29, LLC1, PC3, B16, BG1, MCF7, MDA-MB231, RKO, RKO-AS45-1, SW1417 [SW-1417], SW948 [SW-948], DLD-1, SW480 [SW-480], SW1116 [SW 1116, SW-1116], LS 174T, WiDr, COLO 320DM, COLO 320HSR [COLO 320 HSR], HCT-15, SW403 [SW-403], SW48 [SW-48], HCT-8 [HRT-18], HCT 116, LS123, LS 180, HX [HT1080 xeno], HP [HT1080 poly], Ramos.2G6.4C10, RKO-E6, Hs 255.T, Hs 257.T, Hs 675.T, Caco-2, SK-CO-1, COLO 201, COLO 205, Hs 698.T, LoVo, T84, SW620 [SW-620], SNU-C1, CT26.CL25, CLT 85 [SKI 294/CLT 85], HT 29/36 [SKI 294/HT 29/36], HT 29/26 [SKI 294/HT 29/26], 1116NS-3d, PCA 31.1PCA 33.28, 1116-NS-19-9, 7E12H12, CLT 152 [SKI 294/CLT 152], TAC-1, and GPC-16. The cells to be used for co-culture may include, but are not limited to, fibroblasts, macrophages, endothelial cells, and stem cells. In some embodiments, the cells are breast cancer stem cells. In some embodiments, the cells are colon cancer stem cells.

i. Cell Culture Media

The cells may be cultured in culture medium prior to seeding on the FiSS. The cells seeded on the FiSS may be cultured in culture medium. The culture medium can be altered over the time course of tumoroid formation. For example, the cell culture media can be replaced (such as when passing the cells) or supplemented during culturing. The replacement media can be the same formulation or have a different formulation that the prior media. Other media components can be supplemented to the media during culturing, which can result in a change in the media formulation.

The cell culture media can be a suitable standard base medium that can optionally be supplemented with, without limitation, growth factors, nutrients (e.g. nitrogen, glucose, amino acids), anti-fungals, antibiotics, ions, serum, and/or combinations thereof. Suitable base mediums include without limitation, DMEM, DME, RMPI-1640, and MEM. Others will be appreciated by those in the art.

In some embodiments, the culture media is supplemented with about 5 to about 10% Matrigel. The cell culture media can be supplemented with VEGF, IL6, TGF-β1, or combinations thereof. In some embodiments, the amount of VEGF can be at least 800 pg/mL, can range from about 1 to about 1200 pg/mL, about 100 pg/mL to about 1200 pg/mL, or about 800 pg/mL to about 1200 pg/mL. In some embodiments, the amount of IL6 can be at least 100 pg/mL, can range from about 1 to about 500 pg/mL, about 100 pg/mL to about 500 pg/mL, or about 200 pg/mL to about 500 pg/mL. In some embodiments, the amount of TGF-β1 can be at least 1200 pg/mL and can range from about 1 to about 1800 pg/mL, about 900 pg/mL to about 1800 pg/mL, or about 1200 pg/mL to about 1800 pg/mL.

In some embodiments, the cell culture media is made of a growth media configured to promote growth of the tumoroid and a conditioned media. Formulations for the growth media will be appreciated by those of skill in the art. The conditioned media can be present at a concentration of about 1% to about 99% of the total culture media. In some embodiments the conditioned media is at least 20% of the total culture media. In further embodiments, the conditioned media can be about 20% to about 50% of the total cell culture media.

The conditioned media can be a human mesenchymal stem cell (MSC) conditioned media. MSC conditioned media can be obtained by culturing human MSC cells for one or more passages and collecting the media that the MSC cells were cultured in. In some embodiments, the MSC condition media is obtained from cell culture media collected at passaged 5 and/or passage. The MSC conditioned media can contain molecules and other compounds secreted by the MSC cells. In some embodiments the MSC media can contain VEGF, IL6, TGF-β1, or combinations thereof. In some embodiments, the amount of VEGF in the MSC conditioned media can be at least 800 pg/mL, can range from about 1 to about 1200 pg/mL, about 100 pg/mL to about 1200 pg/mL, or about 800 pg/mL to about 1200 pg/mL. In some embodiments, the amount of IL6 in the MSC conditioned media can be at least 100 pg/mL, can range from about 1 to about 500 pg/mL, about 100 pg/mL to about 500 pg/mL, or about 200 pg/mL to about 500 pg/mL. In some embodiments, the amount of TGF-β1 in the MSC conditioned media can be at least 1200 pg/mL and can range from about 1 to about 1800 pg/mL, about 900 pg/mL to about 1800 pg/mL, or about 1200 pg/mL to about 1800 pg/mL.

ii. Cell Analysis

Any number of cell characteristics may be measured before, during, or after contact with a drug. For example, cell number and/or cell morphology may be measured, counted, or analyzed. The cells may be manually counted. The cells may be counted by an automated system. Flow cytometry may be used to analyze the cells. The cells may be analyzed by spectrophotometry. The cells may be stained. The cells may be stained with dye. The cells may be stained with conjugated antibodies. The cells may be co-stained. The cells may be stained with fluorophores. The cells may be stained with fluorochrome conjugated antibodies.

c. Drugs for Use in Method of Screening

The drug for use in the herein method described of screening may be a candidate drug for the treatment of cancer. The drug may or may not be a chemotherapeutic agent. The drug may be any compound for which data relating to cancer cell treatment is desired. The drug may be an organic small molecule or a protein, for example. The drugs identified by the disclosed methods can be used, for example, to reduce the growth of cancerous tissues or tumors in a subject, kill cancerous tissues or cells in a subject, or arrest the increase in cell number, cell mass, or both, in cancerous tissues or cells in a subject. Drugs suitable for use in the disclosed methods can include the FDA approved NCI Diversity Set V of 1593 compounds (https://dtp.cancer.gov/organization/dscb/obtaining/availableplates.htm), the contents of which are incorporated herein by reference. The drugs for use in the methods described herein can be synthetic or natural. The drug may be a small molecule. The drug may be an anti-mitotic agent. The drug may be a pro-apoptotic agent. The drug may be an alkylating agent. The drug may be an anti-metabolite. The may be an anti-tumor antibiotic. The drug may be a topoisomerase inhibitor. The drug may be a corticosteroid. The drug may be a differentiating agent. The drug may be for hormone therapy. The drug may be for immunotherapy.

The drug may be, but is not limited to, Evista (Raloxifene Hydrochloride), Keoxifene (Raloxifene Hydrochloride), Nolvadex (Tamoxifen Citrate), Raloxifene Hydrochloride, Tamoxifen Citrate, Abitrexate (Methotrexate), Abraxane (Paclitaxel Albumin-stabilized Nanoparticle Formulation), Ado-Trastuzumab Emtansine, Afinitor (Everolimus), Anastrozole, Aredia (Pamidronate Disodium), Arimidex (Anastrozole), Aromasin (Exemestane), Capecitabine, Clafen (Cyclophosphamide), Cyclophosphamide, Cytoxan (Cyclophosphamide), Docetaxel, Doxorubicin Hydrochloride, Ellence (Epirubicin Hydrochloride), Epirubicin Hydrochloride, Eribulin Mesylate, Everolimus, Exemestane, 5-FU (Fluorouracil Injection), Fareston (Toremifene), Faslodex (Fulvestrant), Femara (Letrozole), Fluorouracil Injection, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ibrance (Palbociclib), Ixabepilone, Ixempra (Ixabepilone), Kadcyla (Ado-Trastuzumab Emtansine), Lapatinib Ditosylate, Letrozole, Megestrol Acetate, Methotrexate, Methotrexate LPF (Methotrexate), Mexate (Methotrexate), Mexate-AQ (Methotrexate), Neosar (Cyclophosphamide), Nolvadex (Tamoxifen Citrate), Paclitaxel, Paclitaxel Albumin-stabilized Nanoparticle Formulation, Palbociclib, Pamidronate Disodium, Perj eta (Pertuzumab), Pertuzumab, Tamoxifen Citrate, Taxol (Paclitaxel), Taxotere (Docetaxel), Thiotepa, Toremifene, Trastuzumab, Tykerb (Lapatinib Ditosylate), Velban (Vinblastine Sulfate), Velsar (Vinblastine Sulfate), Vinblastine Sulfate, Xeloda (Capecitabine), Zoladex (Goserelin Acetate), Avastin (Bevacizumab), Bevacizumab, Camptosar (Irinotecan Hydrochloride), Capecitabine, Cetuximab, Cyramza (Ramucirumab), Eloxatin (Oxaliplatin), Erbitux (Cetuximab), 5-FU (Fluorouracil Injection), Fluorouracil Injection, Irinotecan Hydrochloride, Leucovorin Calcium, Lonsurf (Trifluridine and Tipiracil Hydrochloride), Oxaliplatin, Panitumumab, Ramucirumab, Regorafenib, Stivarga (Regorafenib), Trifluridine and Tipiracil Hydrochloride, Vectibix (Panitumumab), Wellcovorin (Leucovorin Calcium), Xeloda (Capecitabine), Zaltrap (Ziv-Aflibercept), and Ziv-Aflibercept.

In some embodiments, the drug may be Actinomycin D. In some embodiments, the drug may be mithramycin A. In some embodiments, the drug may be epirubicin (hydrochloride). In some embodiments, the cancer treatment drug may be daunorubicin (hydrochloride). In other embodiments, the drug may be a drug used for hormone therapy.

The drug may be added to the culture medium in which the FiSS is seeded with cells is present. The drug may be added to the culture medium at varying concentrations. The drug may be added to the culture medium at varying times after the cells are seeded onto the FiSS. The drug may be added immediately after seeding the cells onto the FiSS. The drug may be added 12 hours after seeding the cells on the FiSS. The drug may be added 18 hours after seeding the cells on the FiSS. The drug may be added 24 hours after seeding the cells on the FiSS. The drug may be added 48 hours after seeding the cells on the FiSS. The drug may be in the culture medium for 12 hours before evaluation of the cells. The drug may be in the culture medium for 24 hours before evaluation of the cells. The drug may be in the culture medium for 48 hours before evaluation of the cells. The drug may be in the culture medium for 72 hours before evaluation of the cells.

d. Evaluation of Drug

Half maximal inhibitory concentration, or IC₅₀, is a measurement representing the halfway point in which a compound of interest produces complete inhibition of a biological or biochemical function. This information may be derived based on pharmacological data in reference to a dose-response curve. As the dosage of an inhibitory compound is increased, the biological function it affects decreases. IC₅₀ may be used as a measurement of antagonist, or inhibitory drug potency, as well as a quantification of the toxicological effects of inhibitory compounds. The IC₅₀ may be calculated using Graph Pad Prism.

The drug for cancer treatment may be determined to have a lower IC₅₀ than the IC₅₀ of a control. The drug for cancer treatment may be determined to have a lower IC₅₀ than the IC₅₀ of other drugs tested. The drug selected for cancer treatment may show efficacy against cancer cells at IC₅₀ values of less than about 500 μM. The drug selected for cancer treatment may show efficacy against cancer cells at IC₅₀ values of less than about 250 μM. The drug selected for cancer treatment may show efficacy against cancer cells at IC₅₀ values of less than about 100 μM. The drug selected for cancer treatment may show efficacy against cancer cells at IC₅₀ values of less than about 75 μM. The drug selected for cancer treatment may show efficacy against cancer cells at IC₅₀ values of less than about 50 μM. The drug selected for cancer treatment may show efficacy against cancer cells at IC₅₀ values of less than about 25 μM. The drug selected for cancer treatment may show efficacy against cancer cells at IC₅₀ values of less than about 10 μM. The drug selected for cancer treatment may show efficacy against cancer cells at IC₅₀ values of less than about 5 μM. The drug selected for cancer treatment may show efficacy against cancer cells at IC₅₀ values of less than about 1000 nM. The drug selected for cancer treatment may show efficacy against cancer cells at IC₅₀ values of less than about 750 nM. The drug selected for cancer treatment may show efficacy against cancer cells at IC₅₀ values of less than about 500 nM. The drug selected for cancer treatment may show efficacy against cancer cells at IC₅₀ values of less than about 250 nM. The drug selected for cancer treatment may show efficacy against cancer cells at IC₅₀ values of less than about 200 nM. The drug selected for cancer treatment may show efficacy against cancer cells at IC₅₀ values of less than about 150 nM. The drug selected for cancer treatment may show efficacy against cancer cells at IC₅₀ values of less than about 100 nM. The drug selected for cancer treatment may show efficacy against cancer cells at IC₅₀ values of less than about 90 nM. The drug selected for cancer treatment may show efficacy against cancer cells at IC₅₀ values of less than about 80 nM. The drug selected for cancer treatment may show efficacy against cancer cells at IC₅₀ values of less than about 70 nM. The drug selected for cancer treatment may show efficacy against cancer cells at IC₅₀ values of less than about 60 nM. The drug selected for cancer treatment may show efficacy against cancer cells at IC₅₀ values of less than about 50 nM. The drug selected for cancer treatment may show efficacy against cancer cells at IC₅₀ values of less than about 40 nM. The drug selected for cancer treatment may show efficacy against cancer cells at IC₅₀ values of less than about 30 nM. The drug selected for cancer treatment may show efficacy against cancer cells at IC₅₀ values of less than about 20 nM. The drug selected for cancer treatment may show efficacy against cancer cells at IC₅₀ values of less than about 10 nM. The drug selected for cancer treatment may show efficacy against cancer cells at IC₅₀ values of less than about 5 nM. The drug selected for cancer treatment may show efficacy against cancer cells at IC₅₀ values of less than about 1 nM.

### 3. METHODS OF TREATMENT

The method of treatment may comprise administering, a drug identified in a screen described herein to a subject in need of such treatment. For example, a drug identified in such screen may be useful in the treatment of cancer in a subject from which the cells used in the screen were obtained. For example, the cells may have been obtained from a biopsy of a tumor from the subject. The drug identified may have an optimal IC₅₀ value against the target cells pursuant to the screen. The drugs identified by the methods disclosed herein may treat cancer by targeting sustained proliferative signaling, evasion of growth suppressors, resistance of cell death, enablement of replicative immortality, induction of angiogenesis, and activating invasion and metastasis.

a. Cancer

Cancer is a group of related diseases that may include sustained proliferative signaling, evasion of growth suppressors, resistance to cell death, enablement of replicative immortality, induction of angiogenesis, and the activation of invasion and metastasis. Cancer that can be treated by the disclosed methods, includes, but is not limited to, astrocytoma, adrenocortical carcinoma, appendix cancer, basal cell carcinoma, bile duct cancer, bladder cancer, bone cancer, brain cancer, brain stem glioma, breast cancer, cervical cancer, colon cancer, colorectal cancer, cutaneous T-cell lymphoma, ductal cancer, endometrial cancer, ependymoma, Ewing sarcoma, esophageal cancer, eye cancer, gallbladder cancer, gastric cancer, gastrointestinal cancer, germ cell tumor, glioma, hepatocellular cancer, histiocytosis, Hodgkin lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer and Wilms tumor. In some embodiments, the cancer is breast cancer. In some embodiments, the cancer is colorectal adenocarcinoma.

ii. Breast Cancer

Breast Cancer has claimed the lives of over 40000 women in the United States alone in 2015 according to the Seer Cancer Statistics and the National institutes of Health, which is second only to lung cancer in cancer morbidity and mortality in women. The factors impeding the discovery of an effective treatment option for breast cancer may include; (i) the resilience of the breast cancer stem cells (BCSCs) to avoid death in presence of treatment, (ii) the unavailability of a screening platform to identify drugs that target BCSCs, and (iii) the high cost of discovering and developing a new drug that would show promise in clinical trials.

The methods disclosed herein may include using BCSCs in the screening for drugs for the treatment of cancer. The 3D culture system may include tumoroids that harbor and maintain BCSCs. The 3D culture system may effectively recapitulate the hypoxic and glycolytic microenvironment of breast cancers. The methods disclosed herein may identify Actinomycin D as a drug for the treatment of breast cancer. Actinomycin D may specifically target and down-regulate the stem cell transcription factor Sox2.

In some embodiments, Actinomycin D may be administered to down-regulate Sox2 and thereby eradicate BCSC population in the tumoroids. This decrease has been demonstrated to be via the suppression of Sox2 transcription leading to decrease in Sox2 protein expression. Actinomycin D may intercalate at the GC rich regions of the Sox2 promoter, and lead to down regulation of Sox2.

ii. Colorectal Adenocarcinoma

Colorectal cancer (CRC), a malignancy that develops in the colon or rectum, is the third most commonly diagnosed cancer in men and women in the United States, with approximately 132,700 new cases anticipated in 2015. The five-year survival rate is 92% for stage I CRC which sharply falls to 11% for stage IV. The use of therapeutic agents is often hindered by de novo induction of drug resistance due to the, emergence of epithelial-mesenchymal transition (EMT) and amplification of colorectal stem cell (CSC) population. The activation of EMT in cancer cells is associated with reduced adhesion between cells and enhanced migratory behavior resulting in dissemination of the disease.

The methods disclosed herein may include using CSCs in the screening for drugs for the treatment of colorectal cancer. The 3D culture system may include tumoroids that are enriched for markers of CSCs. The 3D culture system may include tumoroids that are enriched for markers of epithelial to mesenchymal transition. The methods disclosed herein may identify mithramycin A as a drug for colorectal adenocarcinoma treatment. The methods disclosed herein may identify daunorubicin (hydrochloride) as a drug for colorectal adenocarcinoma treatment. The methods disclosed herein may identify epirubicin (hydrochloride) as a drug for colorectal adenocarcinoma treatment.

b. Modes of Administration

Methods of treatment may include any number of modes of administering a drug identified by the screen disclosed herein. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of drug for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire®). In the pharmaceutical composition, the agent may also be dispersed in a microparticle, e.g. a nanoparticulate composition.

For parenteral administration, the drug can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally, for parenteral administration, the agent can be in the form of an aqueous, lipid, oily or other kind of solution or suspension or even administered in the form of liposomes or nano-suspensions.

c. Dosage

The drugs identified by the screen described herein may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of the drug are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of a disclosed drug may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

d. Combination Therapy

Additional therapeutic agent(s) may be administered simultaneously or sequentially. Sequential administration includes administration before or after the drugs identified by the screen described herein. In some embodiments, the additional therapeutic agent or agents may be administered in the same composition as the drug identified by the screen described herein. In other embodiments, there may be an interval of time between administration of the additional therapeutic agent and the drug identified by the screen described herein. In some embodiments, administration of an additional therapeutic agent with a drug identified by the screen described herein may allow lower doses of the other therapeutic agents and/or administration at less frequent intervals. When used in combination with one or more other active ingredients, the drug identified by the screen described herein and the other active ingredients may be used in lower doses than when each is used singly. Accordingly, the drugs identified by the screen may include those that contain one or more other active ingredients. The above combinations include combinations of drug identified by the screen described herein not only with one other active compound, but also with two or more other active compounds. The additional therapeutic agent may lead to a synergistic effect. The synergistic drug combinations may increase therapeutic efficiency and minimize the development of drug resistance. The additional therapeutic agent may lead to an additive effect.

The methods of the invention will be better understood by reference to the following examples, which are intended as an illustration of and not a limitation upon the scope of the invention.

### 4. EXAMPLES

The present disclosure has multiple aspects, illustrated by the following non-limiting examples.

### Example 1. Synthesis of the FiSS Scaffold

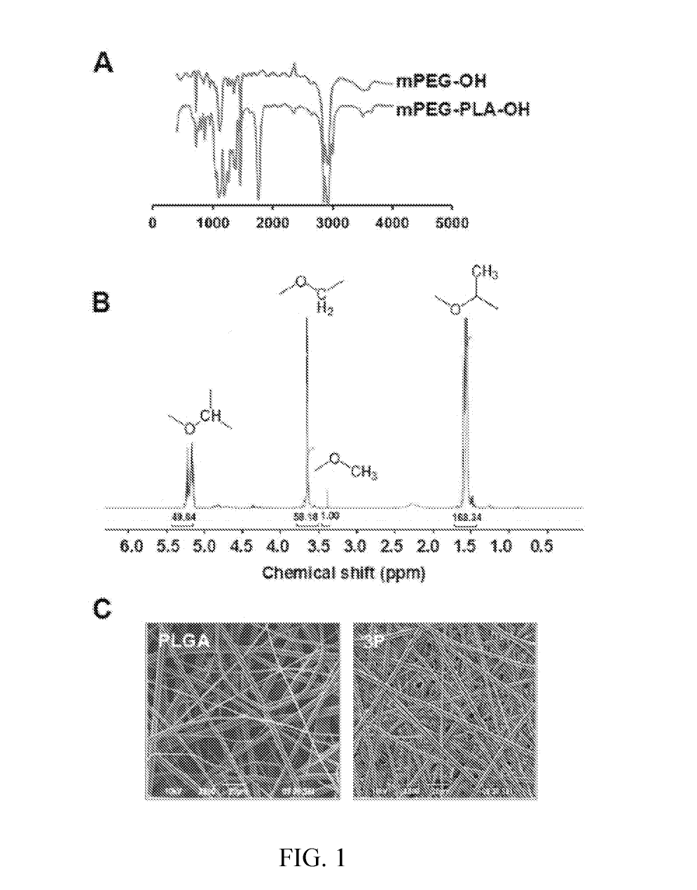

To generate the FiSS, the scaffolds were constructed by electrospinning a solution of the block co-polymer mPEG/LA and PLGA dissolved in appropriate organic solvents. The synthesis of mPEG/PLA was confirmed by FTIR and 1HNMR. FTIR shows strong absorption at 1760 cm21 assigned to the —C═O stretch of PLA. The stretch of the C—O—C band of the mPEG and PLA is shown at 1087 and 1184 cm21, respectively. The peaks at 2850 and 2950 represent —CH2 stretching of the mPEG. (FIG. 1A). The molecular structure of the mPEG-PLA copolymer was characterized by 1H NMR (FIG. 1B). The molecular weight of the PLA block of the mPEG-PLA copolymer was determined to be 23,100 Da using the intensity of the terminal methoxy proton signal at 3.39 ppm as the internal standard. The scaffolds provide good spatial interconnectivity between cells, a high surface-to-volume ratio and good porosity for fluid transport. The parameters that affect the pore size, diameter and thickness of the scaffold include voltage, distance from needle tip to surface of the collecting sheet and concentration of the polymer in the solvent. Scanning electron microscopy (SEM) of the scaffold shows randomly aligned fibers that combine to form a highly porous mesh (FIG. 1C). The diameter of the fibers of the FiSS scaffold ranged from 0.69 to 4.18 μm and of PLGA scaffold ranged 0.61 to 4.95 μm with pores of mainly subcellular sizes (<10 μm).

### Example 2. Characterization of Breast Cancer Stem Cells in Tumoroids

To further investigate the potential of the FiSS for use in cancer drug discovery by characterizing the BCSCs within the tumoroids, MCF-7 cells were seeded onto scaffolds in 6 well plates at 240,000 cells per well. Cells were cultured for 6 days in RPMI media to form primary tumoroids. Primary tumoroids were dissociated by treatment with trypsin and collected for seeding of secondary scaffolds. Cells from primary tumoroids were seeded on to secondary scaffolds in 6 well plates at 240,000 cells per well. These scaffolds were also cultured for 6 days to generate secondary tumoroids. Media was changed on day 4 and media containing either 0 nM, 1.56 nM, or 6.25 nM Actinomycin D was added. Scaffolds were then treated with 1 mL accutase per well for 8 minutes at room temperature on an orbital shaker to dissociate the tumoroids. Scaffolds were washed with PBS to collect cells for analysis by flow cytometry. Secondary tumoroid cells were resuspended in cold FACS buffer (PBS containing 10% FBS and 2.5 mM EDTA) and stained using fluorochrome conjugated antibodies for CD24 and CD44 (Becton Dickenson, and Miltenyi). Isotype control antibodies were used to identify any non-specific binding. Compensation for spectral overlap was performed and data was collected using a BD FACS Canto 2 flow cytometer.

MCF-7 and MCF-7/dox cells were seeded as monolayer and 3D tumoroid cultures in 96 well plates. Actinomycin D was treated at the concentration based on IC₅₀ for 24 hours. Collagenase was used to detach the cells from the plate/scaffold platform. The cell pellet was resuspended in RIPA buffer and vortexed for 30 min followed by centrifugation at 4° C. at 13000 rpm to collect the protein extract. Protein content was determined by using BCA method and followed for western blot procedure. Antibodies for Nanog, Oct 4a, Sox2, and GAPDH/HPRT were obtained from Cell Signaling.

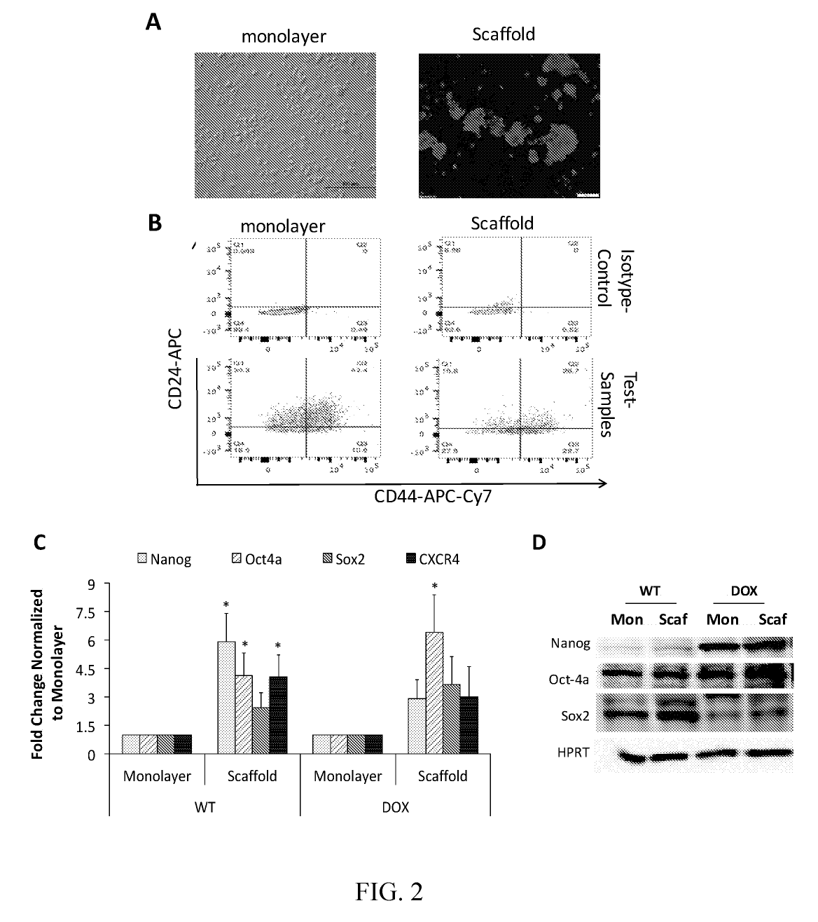

The presence of the BCSCs, as defined by the CD44^{high}CD24^{low} cell population, positively correlates with shorter overall survival in patients, it is imperative to test potential drugs on a platform that amplifies and maintains BSCSs. A nano-fiber scaffold that has been shown to induce growth of 3D tumoroids that mimics in vivo tumors was used. FIG. 2A shows that MCF-7 grew into well-formed single cell tumoroids after 6 days in culture. These tumoroids showed a 3-fold amplification of CD44^{high}CD24^{low} cells when compared to monolayer cells (FIG. 2B). To further confirm, that the growing tumoroids harbored BCSCs, the transcript and the protein levels of the stem cell transcription factors Oct-4, Sox2 and Nanog were analyzed. As seen in FIG. 2C, the transcript levels of all three transcription factors was increased in the tumoroids as compared to the cells growing as a monolayer. Out of the three, only Sox2 protein levels (FIG. 2D) were increased in tumoroids, even though the Oct-4 and Nanog transcripts were statistically higher in the tumoroids compared to the cells growing as a monolayer.

### Example 3. An Increase in BCSCs within the Tumoroids Correlates with Increase in Drug Resistance

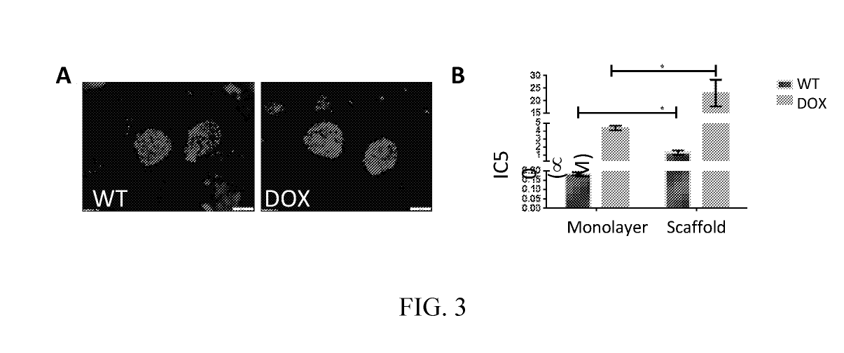

To further validate that stem cell amplification in tumoroids leads to drug resistance, MCF-7 and its syngeneic drug resistant cell line MCF-7/dox were used. MCF-7 tumoroids and MCF-7/dox tumoroids should show induction of BCSCs which in turn should lead to an increased resistance, irrespective of their intrinsic drug sensitivity. MCF-7 and MCF-7/dox formed well-defined tumoroids (FIG. 3A and a doxorubicin dose response curve confirmed that MCF-7/dox (IC₅₀: 4.4±0.28 μM) was 24 fold more resistant than MCF-7 (IC₅₀: 0.18±0.008 μM) in monolayer (FIG. 3B). Due to the enrichment of BCSCs in the tumoroids, doxorubicin showed higher IC₅₀ in the tumoroids (1.31±0.27 μM in MCF-7 vs. 23.09±5.35 μM in MCF-7/dox) while maintaining the differences in drug sensitivity.

### Example 4. Tumoroids Recapitulate Hypoxia when Exposed to Cobalt Chloride

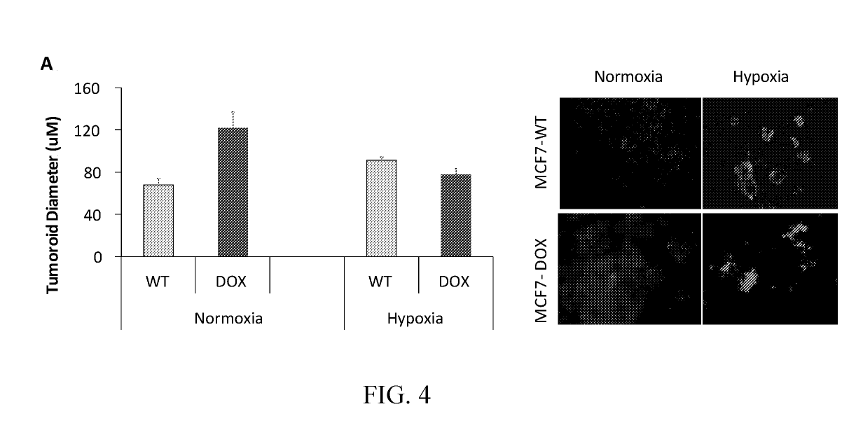

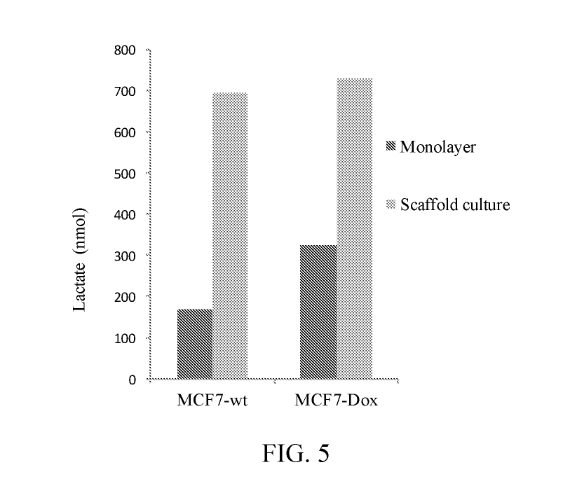

To determine if tumoroids recapitulate hypoxia when exposed to cobalt chloride, the hypoxic region of the 3D scaffold cultures was detected by using Hypoxia detection kit (Enzo Life Sciences) which was designed for functional detection of hypoxia in live cells using fluorescent microscopy. This kit includes fluorogenic probes for hypoxia (red), which takes advantage of the nitroreductase activity present in hypoxic cells by converting the nitro group to hydroxylamine (NHOH) and amino (NH₂) and releasing the fluorescent probe. Briefly, day 6 scaffold culture was incubated with the hypoxia dye for 30 min and incubated at 37° C. followed by PBS washing for two times. Red fluorescence was detected using the fluorescence microscope. Hypoxia is shown to increase drug resistance by inducing a switch to a stem-like phenotype in breast cancer cells, and cobalt chloride (CoCl₂) has been successfully used to mimic hypoxia in monolayer cultures of breast cancer cells. MCF-7 and MCF-7/dox cells were cultured in control (normoxia) vs. CoCl₂ doped FiSS (hypoxia). Tumoroids were allowed to form and it was observed that the MCF-7/dox tumoroids (121.7±15.2 μM) were at least twice as big in size compared to MCF-7 tumoroids (67.8±6.34 μM) in normoxia (FIG. 4A). This difference in size was completely abolished when tumoroids were allowed to form in the presence of CoCl₂ (91.3±3.02 μM in MCF-7 vs. 77.7±5.6 μM in MCF-7/dox). To confirm that treatment with CoCl₂ did indeed lead to depletion of oxygen content within the tumoroids, a red hypoxic dye was utilized. MCF-7/dox tumoroids have an intrinsic hypoxic phenotype compared to the parental MCF-7 tumoroids (FIG. 4B). Exposure to CoCl₂ led to further enhancement of hypoxia in both MCF-7 and MCF-7/dox tumoroids with the MCF-7/dox tumoroids showing the higher intensity of red fluorescence. Keeping with the trend in tumoroids size, MCF-7/dox tumoroids showed higher amounts of lactate release and exposure to CoCl₂ lead to an equivalent increase in lactate in both the MCF-7 and the MCF-7/dox tumoroids (FIG. 5).

### Example 5. High Throughput Screening for Compounds Inducing Cell Death in Breast Cancer

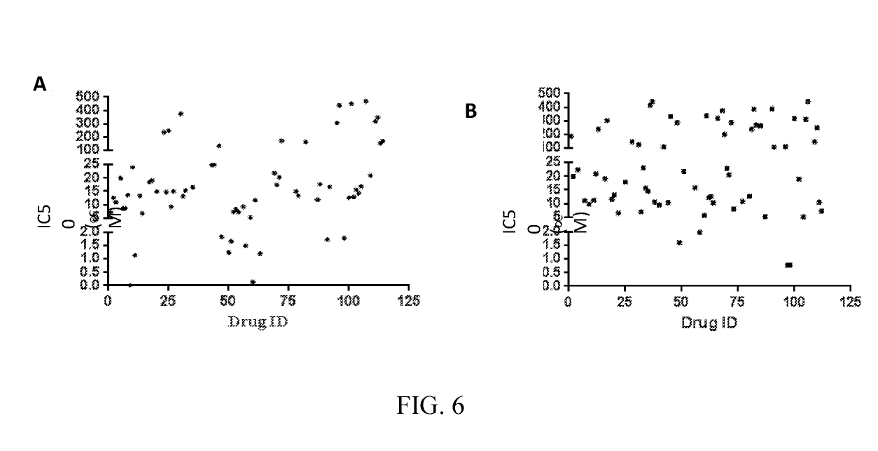

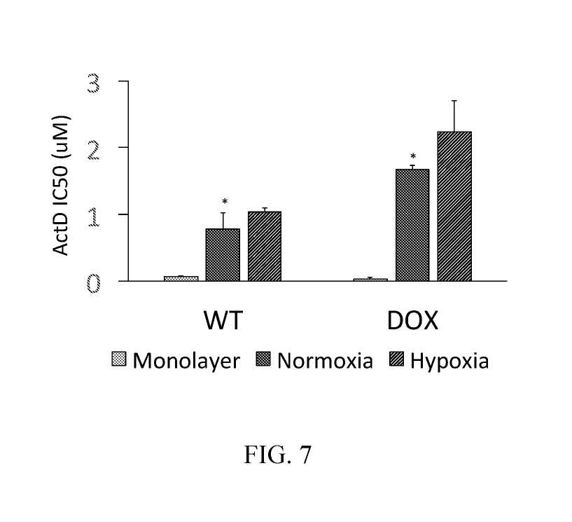

To conduct a high through put screening for compounds that induce cell death in breast cancer, the FDA-approved Oncology Diversity set was obtained from NCI in 96 well plate format with each well containing 20 μl of 10 mM drug stock. All different cell lines were cultured either in regular 96 wells plate without scaffold (served as monolayer) and with FiSS (scaffold culture) in complete medium. For scaffold culture, 75% medium was carefully removed and replaced with 150 μl of fresh medium every alternate day. After five/six days of scaffold culture, cells were stained with NucBlue (Fisher Scientific) to check if tumoroids had formed. Drugs were added in triplicates with varying concentrations. The cells were incubated for 72 hrs in a humidified atmosphere under 5% CO₂ at 37° C., then cell viability was measured using Presto-blue (Fisher Scientific). The IC₅₀ was calculated from the dose response curve using GraphPad Prism Software (version 5.01). MCF-7 ad MCF-7/dox were used in a monolayer in a 96 well plate, and then treated with a NCI Oncology diversity set containing FDA-approved anti-cancer drugs. Each drug was used in a 4 point log-dose from 0.1 to 100 μM. Untreated cells were used as control and the cell death was estimated at the end of 72 hrs post-treatment. FIG. 6 shows the IC₅₀ values of all the drugs tested in MCF-7 (FIG. 6A) and MCF-7/dox (FIG. 6B) cell lines. Actinomycin D, mithramycin A and mitomycin C all showed high potency (low IC₅₀) in both MCF-7 and MCF-7/dox cells. The three hits were validated in a panel of breast cancer cells including MCF-7, MCF-7/dox, MDA-MD-231 and BT474 cells and showed that all three molecules induced cell death in nanomolar concentration (Table 1). Their activity on the 3D cell culture platform was analyzed and it was found that among the three, Actinomycin D showed higher potency in inducing death in tumoroids growing in normoxia or hypoxia. Specifically, Actinomycin D, unlike doxorubicin, had a very minimal increase in IC₅₀ when MCF-7 cells in monolayer was compared to MCF-7/dox monolayer cells (0.07±0.006 μM vs. 0.037±0.021 μM in MCF-7 and MCF-7/dox, respectively) (FIG. 6A). Additionally, unlike mitomycin C or mithramycin A, Actinomycin D was equally effective in inducing cell death in tumoroids grown in normoxic conditions and hypoxic conditions (0.78±0.25 μM vs. 1.04±0.07 μM in MCF-7 and 1.66±0.06 μM vs. 2.24±0.46 μM in MCF-7/dox, respectively) (FIG. 7).

TABLE 1

| Drugs (μM) | MCF7-WT | MCF7-DOX | MDA-MB-231 | BT474 |
|---|---|---|---|---|
| Actinomycin | 0.05 ± 0.01 | 0.05 ± 0.018 | 0.03 ± 0.01 | 0.24 ± 0.03 |
| D | | | | |
| Mithramycin | 0.51 ± 0.17 | 0.69 ± 0.21 | 0.068 ± 0.02 | 0.39 ± 0.06 |
| A | | | | |
| Mitomycin C | 0.17 ± 0.06 | 0.12 ± 0.01 | 0.03 ± 0.01 | 0.03 ± 0.004 |

### Example 6. Actinomycin D Depletes Breast Cancer Stem Cells by Down-Regulating the Expression of Sox2

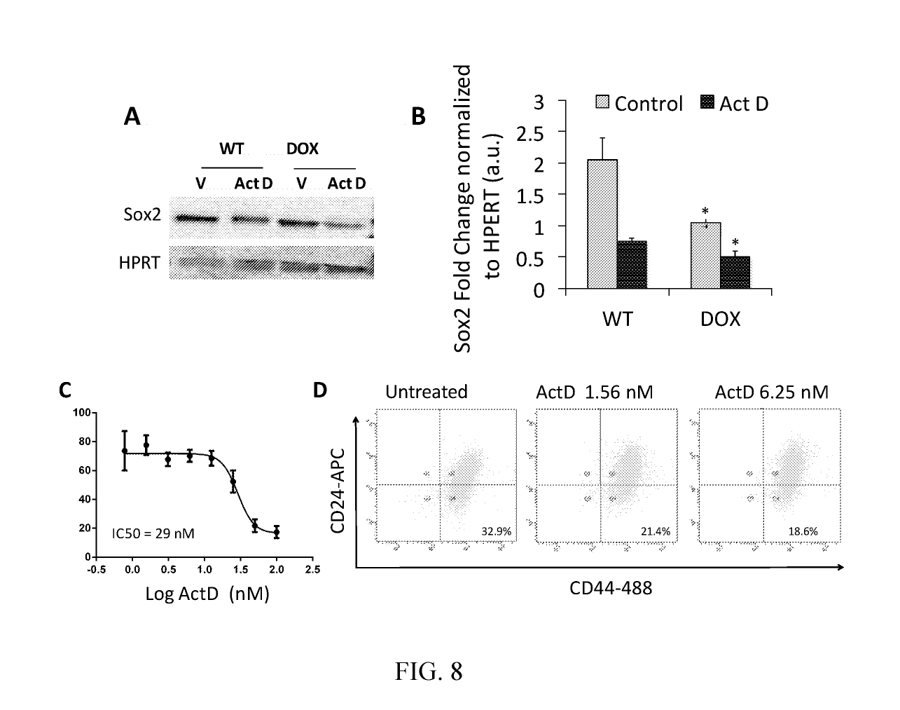

To determine the effects of Actinomycin D on the Sox2 protein levels in both MCF-7 and MCF-7/dox cell lines, MCF7 wt and MCF7-dox cells were seeded as monolayer and 3D tumoroid cultures (both in controls and cobalt chloride (CoCl₂) scaffold cultures) in 96 well plates. Actinomycin D was treated at the concentration based on IC₅₀ for 24 hours. Collagenase was used to detach the cells from the plate/scaffold platform. The cell pellet was then dissolved in trizol and RNA was isolated. RT-PCR was performed using 2 step process namely 1) cDNA synthesis using DNAase-1 treated RNA and 2) PCR was performed using primers of the gene of interest. Tumoroids treated with Actinomycin D resulted in a statistically significant decrease in Sox2 protein expression in both MCF-7 and MCF-7/dox tumoroids (FIG. 8A). The decrease in Sox2 expression was 2 fold in both the cell lines tested when compared to the control untreated tumoroids (2.05 vs. 0.75 pixel density in MCF-7 and 1.05 vs. 0.5 pixel density in MCF-7/dox) (FIG. 8B). Exposure of Actinomycin D to MCF-7 tumoroids decreased cell viability with IC₅₀ 29 nM (FIG. 8C) and the CD44^{high}CD24^{low} as compared to untreated controls (FIG. 8D). Actinomycin D targets and down-regulates the expression of Sox2 resulting in depletion of stem-like cell population, which hampers the breast cancer cell's ability to initiate spheroids.

### Example 7. Determining the IC₅₀ Values in Breast Cancer Cells Post Treatment with Chemotherapeutic Agents

To evaluate IC50 values in breast cancer cells, three human breast cancer cell lines MCF7 (ER+, Her2−), BT474 (ER+, Her2+), and MDA MB-231 (ER−, PR− Her2−) and 4T1 mouse breast cancer cell line were selected. Cells were seeded on monolayer (2D) and on fibrous scaffold (3D) in 96 well plates. Cells were cultured for two days before chemotherapeutic agents were added at varying concentrations. The cells with the agents added were again left to incubate for two more days. Subsequently, cell viability was assayed utilizing Cell titer glo assay according to manufacturer's instruction. The plate was read in the Biotech Automated Plate Reader Gen 5 and the luminescence values (relative to the number of live cells) were obtained for statistical analysis. The IC₅₀ values were calculated using GraphPad Prism and Excel software. Breast cancer cells grow into 3D tumoroids when cultured on the novel cell culture scaffold. Tumoroids showed increased resistance to cell death irrespective of initial drug sensitivity phenotype. Drug resistance in tumoroids co-relates with induction of stem cell markers like the transcription factors Nanog, Sox2 and Oct-4 and cell surface markers like CXCR4 and 7. The 3D cell culture platform can be successfully used to screen for drug sensitivity using a library of compounds. Actinomycin D and mithramycin may be an efficient drug for breast cancer treatment.

### Example 8. Calculating IC₅₀ Values in HT-29 Cells

To calculate IC₅₀ values in HT-29 cells, dual-labeled HT-29 (ATCC® HTB-38) cells were used. HT-29 cells were either grown in the standard 2-dimensional (2D) monolayer culture or in a 3-dimensional (3D) culture system using a novel fibrous scaffold. This 3D cell culture system has previously been shown to assist the formation of tumoroids that enrich for CSCs. The monolayer cells and the 3D tumoroids were independently treated with increasing concentrations of nine FDA approved anti-cancer drugs. After 72 hours post-treatment a Presto Blue Cell Viability Assay (for monolayer) and CellTiter-Glo Viability Assay (for tumoroids) were used to quantitate the drug-induced cell death. The drug treated viability readings were normalized to the viability readings from untreated control. The percent viability was used to calculate the IC₅₀ values of all the nine drugs in HT-29 cells. Screening of nine anticancer drugs on monolayer and scaffold culture revealed mithramycin A, epirubicin (hydrochloride), and daunorubicin (hydrochloride) as the most potent inhibitors of HT-29 cell viability and cisplatin, 6-thioguanine, and cytarabine as the least potent inhibitors. Drug screening on monolayer and scaffold culture has revealed mithramycin A, daunorubicin (hydrochloride), and epirubicin (hydrochloride) as the most potent inhibitors of HT-29 cell viability and cisplatin, 6-thioguanine, and cytarabine as the least potent inhibitors. Treatment of HT-29 tumoroids formed in fibrous scaffold culture resulted in higher IC₅₀ values for 7 of the 9 drugs, indicating heightened drug resistance.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the present disclosure are set out in the following numbered clauses:

Clause 1. A method of screening drugs for cancer treatment, the method comprising: a) growing target cancer cells on a three-dimensional scaffold of fibers, wherein said fibers are formed from a mixture comprising a ratio polyethylene glycol-polylactic acid block copolymer (PEG-PLA) and a poly(lactic-co-glycolic acid) (PLGA); b) contacting at least one drug to the cells; and c) measuring IC₅₀ values of the at least one cancer drug.

Clause 2. The method of clause 1, wherein the fibers are randomly oriented.

Clause 3. The method of clause 1, wherein the ratio of PEG to PLA is from about 1:2 to about 1:20.

Clause 4. The method of clause 3, wherein the ratio of PEG to PLA is from about 1:4 to about 1:10.

Clause 5. The method of clause 4, wherein fiber diameter ranges from about 0.3 μm to about 10 μm.

Clause 6. The method of clause 1, wherein the scaffold comprises pores having a diameter between about 5 mm to about 20 μm.

Clause 7. The method of clause 6, wherein the scaffold comprises pores having a diameter of less than about 10 μm.

Clause 8. The method of clause 1, wherein the PEG has a molecular weight of about 2 kDa.

Clause 9. The method of clause 1, wherein the PLGA has a lactic acid:glycolic acid ratio of between about 75:25 to about 95:5.

Clause 10. The method of clause 1, wherein the PLGA has a lactic acid:glycolic acid ratio of about 85:15.

Clause 11. The method of clause 1, wherein the fibers of the scaffold are formed by electrospinning.

Clause 12. The method of clause 1, wherein the target cancer cells obtained are from a tumor biopsy.

Clause 13. The method of clause 1, wherein the target cancer cells are co-cultured cells.

Clause 14. The method of clause 12, wherein the tumor biopsy is from a subject prior to treatment for cancer or a subject undergoing treatment for cancer.

Clause 15. The method of clause 12, wherein the tumor biopsies are from a subject with breast cancer.

Clause 16. The method of clause 12, wherein the tumor biopsies are from a subject with colorectal adenocarcinoma.

Clause 17. The method of clause 1, wherein higher IC₅₀ values indicate drug resistance.

Clause 18. The method of clause 12, further comprising administering the at least one drug to the subject from which the tumor biopsy was derived, wherein the drug has a lower IC50 value in comparison to other drugs screened.

Clause 19. The method of clause 1, wherein the drug is selected from the group comprised of Actinomycin D, mithramycin, epirubicin, and daunorubicin, or a pharmaceutically acceptable excipient.

Clause 20. The method of clause 1, wherein the cancer is breast cancer.

Clause 21. The method of clause 1, wherein the cancer is colorectal adenocarcinoma.

Clause 22. The method of clause 1, wherein two or more drugs are contacted to the cells.

Clause 23. The method of clause 22, wherein the drugs combined have an IC₅₀ value that indicates additive effects of the drugs.

Clause 24. The method of clause 22, wherein the drugs combined have an IC₅₀ value that indicates synergistic effects of the drugs.

What is claimed is:

1. A method of screening drugs for cancer treatment, the method comprising:
a) growing target cancer cells on a three-dimensional scaffold of fibers, wherein said fibers are formed from a mixture comprising a ratio polyethylene glycol-polylactic acid block copolymer (PEG-PLA) and a poly(lactic-co-glycolic acid) (PLGA);
b) contacting at least one drug to the cells; and
c) measuring IC₅₀ values of the at least one cancer drug.

2. The method of claim 1, wherein the fibers are randomly oriented.

3. The method of claim 1, wherein the ratio of PEG to PLA is from about 1:2 to about 1:20.

4. The method of claim 3, wherein the ratio of PEG to PLA is from about 1:4 to about 1:10.

5. The method of claim 4, wherein fiber diameter ranges from about 0.3 μm to about 10 μm.

6. The method of claim 1, wherein the scaffold comprises pores having a diameter between about 5 mm to about 20 μm.

7. The method of claim 6, wherein the scaffold comprises pores having a diameter of less than about 10 μm.

8. The method of claim 1, wherein the PEG has a molecular weight of about 2 kDa.

9. The method of claim 1, wherein the PLGA has a lactic acid:glycolic acid ratio of between about 75:25 to about 95:5.

10. The method of claim 1, wherein the PLGA has a lactic acid:glycolic acid ratio of about 85:15.

11. The method of claim 1, wherein the fibers of the scaffold are formed by electrospinning.

12. The method of claim 1, wherein the target cancer cells obtained are from a tumor biopsy.

13. The method of claim 1, wherein the target cancer cells are co-cultured cells.

14. The method of claim 12, wherein the tumor biopsy is from a subject prior to treatment for cancer or a subject undergoing treatment for cancer.

15. The method of claim 12, wherein the tumor biopsies are from a subject with breast cancer.

16. The method of claim 12, wherein the tumor biopsies are from a subject with colorectal adenocarcinoma.

17. The method of claim 1, wherein higher IC₅₀ values indicate drug resistance.

18. The method of claim 12, further comprising administering the at least one drug to the subject from which the tumor biopsy was derived, wherein the drug has a lower IC₅₀ value in comparison to other drugs screened.

19. The method of claim 1, wherein the drug is selected from the group comprised of Actinomycin D, mithramycin, epirubicin, and daunorubicin, or a pharmaceutically acceptable excipient.

20. The method of claim 1, wherein the cancer is breast cancer.

21. The method of claim 1, wherein the cancer is colorectal adenocarcinoma.

22. The method of claim 1, wherein two or more drugs are contacted to the cells.

23. The method of claim 22, wherein the drugs combined have an IC₅₀ value that indicates additive effects of the drugs.

24. The method of claim 22, wherein the drugs combined have an IC₅₀ value that indicates synergistic effects of the drugs.
